(19) United States
(12) Patent Application Publication
(10) Pub. No.: US 2018/0277258 A1
(43) Pub. Date: Sep. 27, 2018

(54) SYSTEM FOR REMOTE GUIDANCE OF HEALTH CARE EXAMINATIONS

(71) Applicant: TELSHUR INC., Vancouver, WA (US)

(72) Inventor: Sanjiv PANWALA, Washougal, WA (US)

(21) Appl. No.: 15/990,183

(22) Filed: May 25, 2018

Related U.S. Application Data
(63) Continuation of application No. PCT/US2017/045964, filed on Aug. 8, 2017.
(60) Provisional application No. 62/372,248, filed on Aug. 8, 2016.

(51) Int. Cl. G16H 80/00; G16H 70/20; G06F 17/30

(52) U.S. Cl. CPC G16H 80/00; G06F 17/30864; G16H 70/20

(57) ABSTRACT

A distributed computer system for conducting remote medical examinations may include a queryable database comprising a plurality of examination components, each of which is related to at least one respective physiological system and/or at least one medical diagnosis. The queried result may be further narrowed automatically, based on additional information such as known best practices, and/or manually by a user having sufficient system credentials. The system may send the resulting examination procedure to a target presenter at a target facility, and may provide a communication channel for real-time communication between a provider and the presenter, e.g., during subsequent examination of a patient at the target facility.

### FIELD

This disclosure relates to systems and methods for remotely providing health care, such as in the fields of telemedicine or telehealth. More specifically, the disclosed embodiments relate to systems and methods for generating, communicating, and supervising examinations over a data processing network.

### INTRODUCTION

Access to medical professionals for patient care needs is a challenge faced by many facilities, including rural and remote hospitals, clinics, nursing homes, and other facilities. For example, certain facilities may not staff a physician on the night shift, due to funding, staffing shortages, or other considerations. In some examples, such as nursing homes and some international facilities, staffing may not include physicians and/or specialists, by design. Nevertheless, medical problems still occur whether or not an adequately trained staff member is present. In response, the field of telemedicine or telehealth has become more and more prevalent, wherein a doctor or other provider at one facility can remotely assist a trained assistant (known as a presenter) at another facility. For example, a provider in a first location (e.g., an urban hospital) may engage in a videoconferencing session with a patient and a nurse at a second location (e.g., a critical access hospital (CAH), nursing home, or remote location). During the videoconference, the provider may interact with the patient verbally, and may instruct the nurse to perform certain actions to aid in diagnosis and/or treatment of the patient. Known methods for providing health care in this manner suffer from various drawbacks, such as undertrained local personnel, inconsistent protocols, inadequate interfaces for remote conferencing, and ad hoc reliance on provider expertise and experience.

### SUMMARY

The present disclosure addresses the issues outlined above, and provides systems, apparatuses, and methods relating to remote examination and physical assessment systems for use in telehealth and telemedicine.

In some embodiments, a distributed data processing system for providing examination guidance remotely may include: a processor; a memory in communication with the processor; and a set of instructions stored in the memory and executable by the processor to: query, in response to a search request from a first user at a first computer of the system, a data store containing a plurality of examination components, each examination component relating to at least one respective physiological system and to at least one respective diagnosis; return a recommended subset of the examination components based on the search request; generate, in response to selection of one or more of the examination components from the recommended subset by the first user, an examination procedure comprising the selected examination components; and communicate the examination procedure to a second user at a second computer of the system.

In some embodiments, a method for remotely providing examination guidance may include: establishing a first communication link between a first computer and a node in a computer network; querying a database containing a plurality of examination components using the first computer, each of the examination components relating to at least one respective physiological system and to at least one respective medical diagnosis, by providing search criteria comprising a selected physiological system or a selected medical diagnosis or both; narrowing a subset of the examination components returned from the database to generate an examination procedure comprising selected examination components from the subset; establishing a second communication link between a second computer of the computer network and the node; and communicating the examination procedure to the second computer via the node.

In some embodiments, a method for remotely providing examination guidance may include: establishing a first communication link between a first computer in a first facility and a network-instantiated presentation layer in a computer network; querying a database containing a plurality of examination components using the first computer, each of the examination components relating to at least one respective physiological system and to at least one respective medical diagnosis, by providing search criteria comprising a selected physiological system or a selected medical diagnosis or both; narrowing a subset of the examination components returned from the database to generate an examination procedure comprising selected examination components from the subset; establishing a second communication link between a second computer of the computer network and the presentation layer, the second computer disposed in a second facility; communicating the examination procedure to the second computer; and providing a real-time communication channel between the first computer and the second computer via the presentation layer.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a schematic diagram of an illustrative system for remote guidance of health care examinations, in accordance with aspects of the present disclosure.

FIG. 2 is a schematic diagram of an illustrative database suitable for use in the system shown in FIG. 1.

FIG. 3 is a flow chart depicting steps in an illustrative method for remotely guiding (as a provider) a health care examination, according to the present teachings.

FIG. 4 is a flow chart depicting steps in an illustrative algorithm for dynamically generating an examination procedure, according to the present teachings.

FIG. 5 is a flow chart depicting steps in an illustrative method for locally executing (as a presenter) a health care examination, e.g., under the guidance of a remote expert, according to the present teachings.

FIG. 6 is a schematic diagram of an illustrative data processing system, suitable for implementing aspects of the present disclosure.

FIG. 7 is a schematic diagram of an illustrative data processing network, suitable for implementing aspects of the present disclosure.

### DESCRIPTION

Various aspects and examples of a remote examination system having automatic exam generation and communication functionality, as well as related methods, are described below and illustrated in the associated drawings. Unless otherwise specified, a remote examination system and/or its various components may, but are not required to, contain at least one of the structure, components, functionality, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

The term “examination” may include any form of assessment or evaluation relating to the physical and/or mental condition of a patient, including examination procedures involving the use of medical equipment. Such medical equipment may include a stethoscope, an oxygen saturation monitor, ultrasound, and/or the like.

### Overview

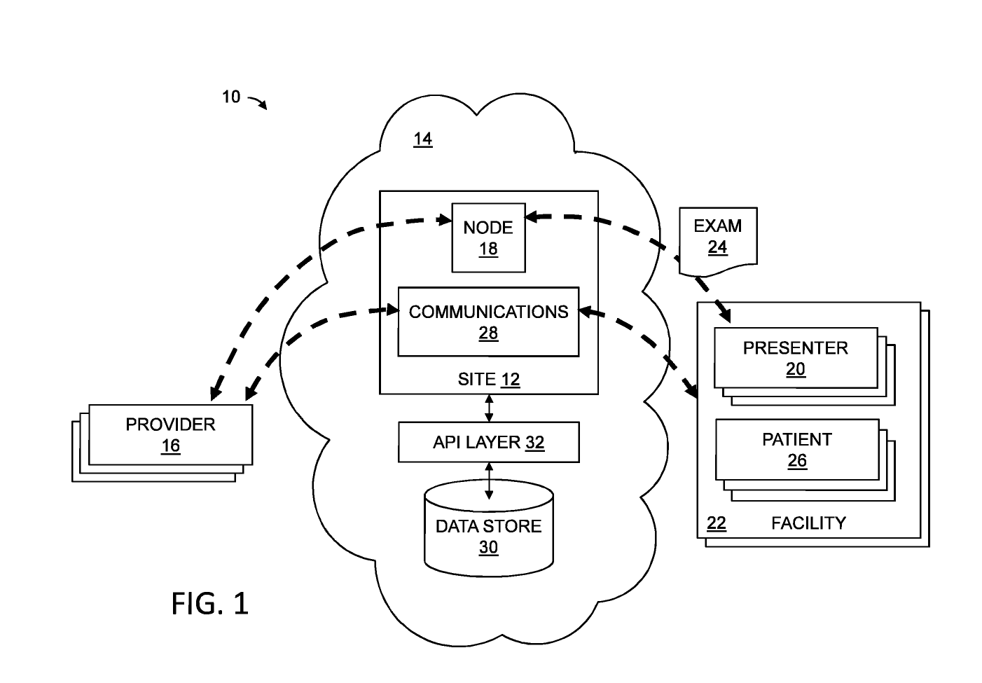

In general, and with reference to FIG. 1, a remote examination system according to the present teachings may include a network-based site accessible by providers and presenters for assembly and communication of examination procedures. As depicted schematically in FIG. 1, a remote examination system 10 may include a site 12 (e.g., a website or local area network presentation node) implemented over a computer network 14 (e.g., the Internet, a LAN), which may be referred to as “the cloud.”

As indicated in FIG. 1, one or more providers 16 may establish a link to a node 18 on site 12, e.g., using login credentials. Similarly, one or more presenters 20 at facility(ies) 22 may establish a link to node 18, e.g., by using their own login credentials. Through this arrangement, the one or more presenters can establish an indirect connection to provider(s) 16 via node 18. An examination procedure 24 may be generated by site 12 at the direction of provider 16, and communicated to presenter 20 at facility 22 via node 18. The examination procedure may be utilized by presenter 20, either independently or in cooperation with provider 16, to examine a patient 26 collocated with the presenter. When performed independently, information learned or determined from the examination may be communicated to the provider (e.g., asynchronously) via any suitable method or format (e.g., written, verbal, video, etc.).

When performed in cooperation with provider 16, the provider may remotely supervise and/or direct the examination of patient 26, at least in part using examination procedure 24. This supervision may be facilitated or enhanced by direct communications between the provider and the facility. Specifically, provider 16 in one location may be in textual, voice, image, and/or video communication with presenter 20 and patient 26 in another location, through a communications module 28 associated with site 12. For example, provider 16 may be “present” at the patient's examination through a video chat session. Providers may make exam modifications based on real-time assessment or changes in patient condition. Providers and presenters may view exam component information, such as explanations of how to perform the examination component, what is tested by the component, etc., at any time during the examination session.

Providers 16 may include any suitable provider of health care or the like, such as physicians, medical doctors, osteopaths, chiropractors, veterinarians, dentists, surgeons, physician's assistants (PAs), specialists, etc. Presenters 20 may include any suitably trained individual capable of carrying out the examination procedures under a provider's supervision, such as health care technicians, nurses, nurse practitioners, PAs, physicians having a different specialty or level of training than the provider, etc.

Site 12 may include any suitable network-instantiated presentation layer configured to provide a user interface for underlying functionality. Site 12 may include supporting layers, such as one or more data stores 30 for storing information relating to users, examination components, diagnoses, and the like, as further described below. An interface layer 32 may provide communication between site 12 and data store 30. Interface layer 32 may include an application programming interface (API). Site 12 may query the data store for user login purposes, generation of examination procedures, and the like. In combination, interface layer 32 and data store 30 may be referred to as the site's “backend.”

Although systems and methods are presented in the context of a medical examination or similar situation, aspects of the present disclosure may be applied or utilized in any suitable context where a remote expert is guiding a lesser-trained individual in the local execution of a process or method. Any suitable setting may be used, such as a clinic, operating room, examination facility, repair shop, etc.

Aspects of remote examination systems disclosed herein may be embodied as a computer method, computer system, or computer program product. Accordingly, aspects of the remote examination system may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the remote examination system may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of the remote examination system may be written in one or any combination of programming languages, including an object-oriented programming language such as Java, Smalltalk, C++, and/or the like, and conventional procedural programming languages, such as C. Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the remote examination system are described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be provided to a processor of a general purpose computer, special purpose computer, and/or other programmable data processing apparatus to produce a machine, such that the instructions, which execute via the processor of the computer or other programmable data processing apparatus, create means for implementing the functions/acts specified in the flowchart and/or block diagram block(s).

These computer program instructions can also be stored in a computer-readable medium that can direct a computer, other programmable data processing apparatus, and/or other device to function in a particular manner, such that the instructions stored in the computer-readable medium produce an article of manufacture including instructions which implement the function/act specified in the flowchart and/or block diagram block(s).

The computer program instructions can also be loaded onto a computer, other programmable data processing apparatus, and/or other device to cause a series of operational steps to be performed on the device to produce a computer-implemented process such that the instructions which execute on the computer or other programmable apparatus provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the remote examination system. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

### Examples, Components, and Alternatives

The following sections describe selected aspects of exemplary remote examination systems, as well as related systems and/or methods. The examples in these sections are intended for illustration and should not be interpreted as limiting the entire scope of the present disclosure. Each section may include one or more distinct embodiments or examples, and/or contextual or related information, function, and/or structure.

### A. Illustrative Database

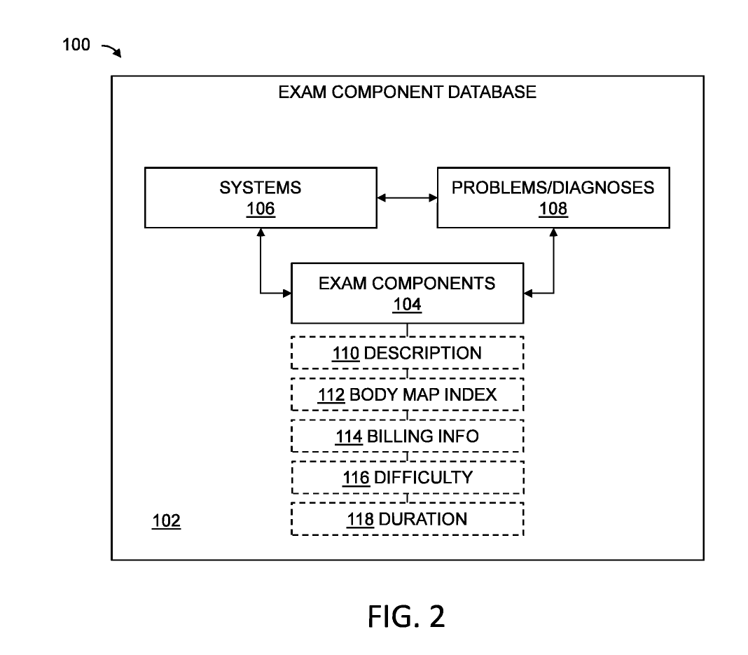

As shown in FIG. 2, this section describes a database system 100 suitable for use in a remote examination system, such as system 10 described above. Database system 100 is an example of data store 30.

Database system 100 includes one or more databases 102, which may include any suitable relational database configured to store information and respond to queries regarding medical examinations. As such, database 102 includes data records corresponding to examination components 104 (also referred to as exam elements), physiological systems 106, and problems or diagnoses 108.

Examination components 104 may include any suitable granular elements combinable to generate a set of examination instructions (e.g., exam 24). Examination components 104 may each include one or more examination steps configured to assess a selected physiological system or determine the likelihood of a selected diagnosis. For example, examination components 104 may include items such as a cardiac auscultation, gait assessment, testing for ability to speak in full sentences, palpation for inguinal hernia, evaluation of the scalp, and the like. Combined into a set of examination instructions for the presenter, the components may help a provider diagnose and/or treat a patient.

Each of the exam components may be related to one or more physiological systems 106. For example, general examination of the skin may be related to the dermatological system. Each system may be further subdivided, such that an exam component may be related to a physiological system and to a subsystem of that system. For example, evaluation of nystagmus may be related to the motor neuron subsystem of the neurological system. In other words, physiological systems 106 may include a hierarchy of systems.

Each exam component 104 may be related to one or more diagnoses 108. Diagnoses 108, also referred to as problems or conditions, may include any suitable medical, physiological, psychiatric, or other condition with which the patient may be diagnosed. Each exam component may tend to indicate, rule out, or otherwise provide clues regarding one or more such conditions. Examples of diagnoses 108 may include Sjogren's disease, rheumatoid arthritis, stroke, Parkinson's, etc.

Exam components 104, systems 106, and diagnoses 108 may all be related to each other within database 102. As described above, each exam component may be associated with one or more physiological systems and with one or more diagnoses. Similarly, each diagnosis may be associated with one or more physiological systems and components, and each physiological system may be associated with one or more components and diagnoses. Accordingly, these associations may be identified and stored in database 102. Said another way, the exam component data 104 in database 102 may be stored in association with physiological systems data 106 and diagnosis data 108. This permits automatic association of the information, and facilitates queries of the database. For example, retrieval of a selected system may automatically retrieve all exam components associated with that system. In another example, retrieval of a selected diagnosis may automatically retrieve all exam components associated with that diagnosis (e.g., associated with the differential diagnosis of that problem).

Additional fields may be present in database 102. For example, exam components 104 may have related fields, such as a description 110 of the exam component. Description 110 may include any suitable textual, audio, visual, video, and/or other information explaining, demonstrating, and/or providing instructions regarding the exam component. For example, description 110 may include a textual description of the component, an explanation of the information to be gleaned from the component, and a short video or diagram showing how to perform the exam component. In some examples, description 110 may include information regarding pathophysiology of the exam component and what it tests, and/or reference material or reference citations associated with the component.

Exam components 104 may have other related fields, such as a body map index 112, billing information 114, difficulty information 116, and/or duration information 118. Body map index 112 may include any suitable information relating to a portion of the patient's body where the exam component is substantially directed (e.g., head, torso, foot, etc.) This information may be utilized in arranging exam components into an efficient or intuitive ordering for the presenter.

Billing information 114 may include information regarding the financial billing of the exam components. Examinations in general may require a certain number or total value of the exam components to permit billing or to result in an exam that is billable at a selected level, with respect to insurance. In some examples, this is determined by the number of “points” associated with an examination. Each activity (i.e., component) within the examination has a point value, and the point value of the overall examination is the sum of those point values. Accordingly, each component in database 102 may have a field that stores the number of points associated with that component.

Difficulty information 116 may include a difficulty rating and/or a required skill or certification level. This information may be utilized to suggest possible presenters for a particular examination, to flag an examination as beyond the capacity of the current presenter, or to determine the overall difficulty of the examination as a whole.

Duration information 118 may include the expected length of time associated with the exam component, such that an overall length of the examination can be estimated. This may also facilitate a provider substituting or eliminating certain lower priority exam components when limited time is available.

Other information available through the database 102 may include current test results, known preexisting conditions of the patient, and the like, which may negate (or add) the need for certain exam components. This type of information may be used by the provider in guiding the examination and/or making a diagnosis, or by the system to annotate or automatically disable the selection of exam components that may be redundant, unnecessary, impossible, or inadvisable.

B. Illustrative Method from the Provider's Point of View

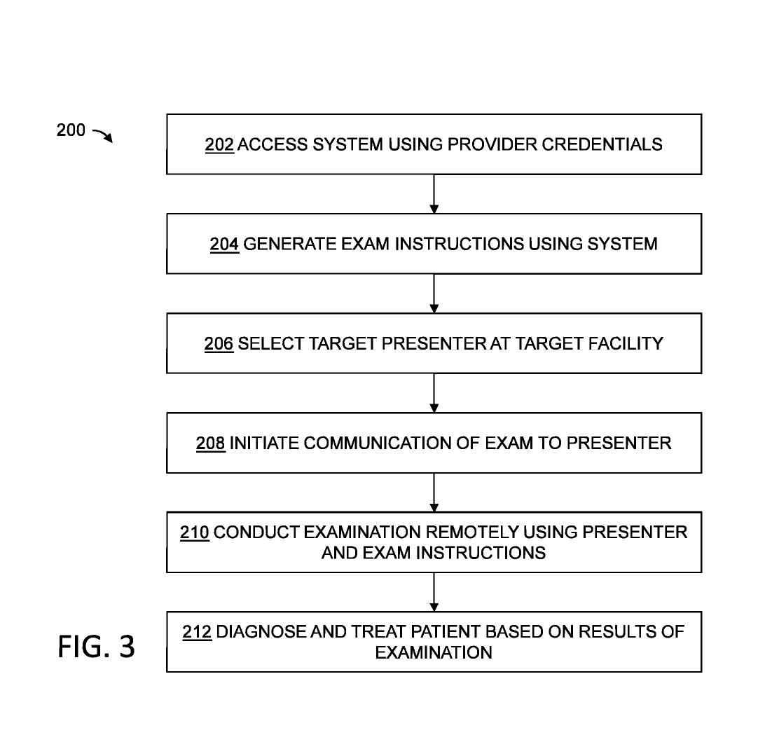

This section describes steps of an illustrative method 200 for utilizing system 10 and database 100 as provider 16; see FIG. 3. Aspects of system 10 and database 100 may be utilized in the method steps described below. Where appropriate, reference may be made to previously described aspects and systems that may be used in carrying out each step, as well as to other methods described herein. These references are for illustration, and are not intended to limit the possible ways of carrying out any particular step of the method.

FIG. 3 is a flowchart illustrating steps performed in an illustrative method, and may not recite the complete process or all steps of the method. Although various steps of method 200 are described below and depicted in FIG. 3, the steps need not necessarily all be performed, and in some cases may be performed simultaneously or in a different order than the order shown.

At step 202, the provider may access the system using provider credentials. Logging into the system, the provider is granted access to interact with the system and to communicate with the facilities assigned to (or otherwise associated with) the presenter. As a provider, the user is granted permission to generate and communicate examination instructions.

At step 204, the provider may generate a final version of the examination instructions using the system. Further details regarding generation of the examination instructions are provided below, with respect to method 300. The provider may be informed (e.g., by accessing medical records, by another provider or site staff, by the presenter, by an email from another party, etc.) of the general complaint(s) and/or symptoms of a patient. Based on that information, the provider may search for or select a particular physiological system 106 and/or a possible diagnosis. The examination system 10 will list all of the exam components associated with that diagnosis or physiological system. The provider will then select the exam components he or she wants performed by the presenter, and a final examination will be created. The final examination document may comprise a listing of the selected exam components, as well as links to descriptions and/or video demonstrations, or the like. The provider may also add further comments to any of the exam components, or provide additional specific requests to the presenter, e.g., in a text field, either associated with a particular exam component or independent from the list of exam components.

At step 206, a target presenter may be selected at the target facility, if not already established. This step may connect the provider to the presenter through a node of the site. For example, the presenter may already be logged into the system and may have requested assistance. The provider may connect to the presenter through the system to provide the assistance requested. This connection is separate from and in addition to a standard communication channel used for telemedicine and involving the patient, such as a live video session. Providing a second connection exclusively between the provider and the presenter may improve patient privacy, provide a better avenue for certain presenter/provider discussions, and/or prevent errors caused by confusion regarding the intended recipient of an examination document, etc.

At step 208, the exam instructions may be communicated to the presenter through the system. The presenter will be alerted to the incoming exam document. Note that the examination procedure/instructions are referred to here as a “document” for convenience. However, the examination procedure may take any suitable form, such as by displaying the instructions on the user's GUI, providing an audible voice reading of the selected components, as an attachment to a system message or email, through an animated presentation, or the like, or any combination of these. In some examples, the exam document may be prepared and sent before the presenter is available, in which case it may be held by the system until retrieved by the presenter.

At step 210, the examination may be performed remotely, by the presenter either independently or with the provider (or a combination of both), using the exam document generated in step 204. Examination of the patient may include establishing a (e.g., real time) mode of communication, such as a video chat, instant messaging, voice, and/or visual image mode of communication, e.g., over the exclusive link between the provider and presenter. For example, performing step 208 may trigger the system to establish a video chat session between the provider and the selected presenter. In some examples, multiple communication sessions may be opened simultaneously. For example, a typical telemedicine video chat session may be used for general communication between provider, presenter, and patient, while an instant messaging session may be used to convey other information privately between the provider and presenter. Examination may be performed independently, using the exam document generated in step 204. In such cases, the presenter may communicate results back to the provider after the examination is partially or fully completed. Examination may be an iterative process, in which the provider modifies the examination instructions based on real-time feedback and communications between the provider, presenter, and/or patient.

At step 212, the provider may diagnose and/or treat the patient based on the results of the examination. For example, a differential diagnosis may be performed and a course of treatment or medication may be prescribed.

C. Illustrative Method from the System's Perspective

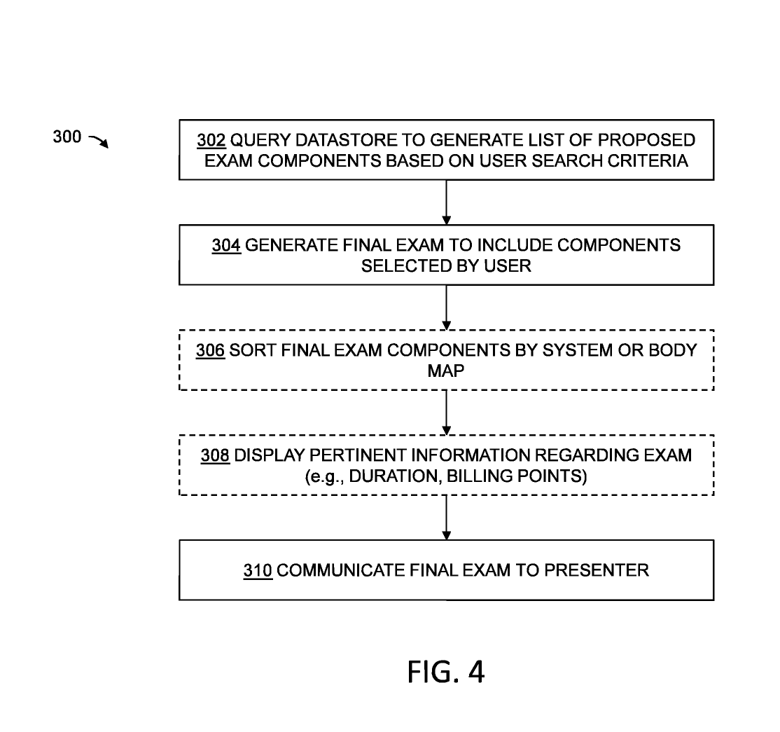

This section describes steps of an illustrative method 300 for generating an examination document, such as exam 24; see FIG. 4. Aspects of system 10 and database system 100 may be utilized in the method steps described below. Where appropriate, reference may be made to previously described components and systems that may be used in carrying out each step, as well as other methods described herein. These references are for illustration, and are not intended to limit the possible ways of carrying out any particular step of the method.

FIG. 4 is a flowchart illustrating steps performed in an illustrative method, and may not recite the complete process or all steps of the method. FIG. 4 depicts multiple steps of a method, generally indicated at 300, which may be performed in conjunction with system 10, database system 100, and/or method 200, according to aspects of the present disclosure. Although various steps of method 300 are described below and depicted in FIG. 4, the steps need not necessarily all be performed, and in some cases may be performed simultaneously or in a different order than the order shown.

At step 302, the system may query the data store (e.g., database 102, using API 32) to generate a list of proposed and/or relevant examination components (e.g., components 104), based on user search criteria, and/or based on a set of components previously selected and/or stored by the user. For example, a provider may search for desired exam components by selecting or interactively searching for a physiological system, diagnosis, or diagnosis group, or a specific component or component group. In some examples, the user may have previously created a “favorites” or “always include” list of exam components, or a template, which may be recalled by the system such that the searched-for components are added to the existing list. Searching and selecting may be performed through a graphical user interface (GUI) of the site, e.g., using a mouse or touch interface, entering text, and/or using voice commands.

Querying the data store may return a list of exam components related to the selected or searched term(s). These components may be presented to the user/provider, and the user/provider may select all or a subset of the examination components to be included in the final examination document, e.g., based on the user's experience and/or other criteria. Patient-related information, such as existing conditions and known characteristics or lab results, may be utilized by the provider and/or system to further filter or suggest exam components.

In some examples, data from system 10 may be accumulated over time, across users, and/or across use cases, and suitable methods such as artificial intelligence, machine learning, data analysis, and/or the like, may be used (e.g., in combination with external sources of data) to ascertain patterns and best practices. This information and analysis may be utilized, e.g., on an ongoing basis, to automatically improve the system. For example, digital presentation of clinical processes and exam components in step 304 may be improved based on use patterns of certain diagnostics and assessments. A specific example of this would be the preferential presentation by system 10 of assessments that show a preferential use based on provider characteristics (e.g., cardiologists) and/or condition (e.g., heart disease). In some examples, assessments, procedures, diagnostics, or clinical protocols may be suggested by the system to the provider based on clinical outcomes data. Specifically, external data on patient outcomes may be used to suggest diagnostics or assessments that are associated with better patient outcomes. In some examples, the most efficient use of medical diagnostics and assessments may be suggested, based on user and external data. For example, system 10 may suggest exams or diagnostics based on collected information relating to their relative value compared to other options.

As mentioned above, the user querying the data store may create and save one or more personal exam component lists, based on personal preference, which can be used in assembling the final examination document.

At optional step 306, the system may sort (i.e., order) the selected exam components based on certain criteria. A default sorting scheme may be used, with alternative sorting schemes selectable by the user. For example, exam components may be sorted by physiological system, by component name (e.g., alphabetically), by relative importance, by duration, by difficulty, and/or by body map index. Sorting by body map index may be useful, in that the exam components can be listed in a head-to-toe or other logical order, which can be more intuitive for the presenter. Sorting the exam components according to one or more of these schemes may improve examination efficiency.

As the user selects or deselects components, optional step 308 may include the system updating the displayed data to include information describing the overall examination, such as total duration, billing points/level, and/or difficulty. The provider may use this information to further modify the set of components, e.g., to ensure the duration is less than a selected time limit or to adapt difficulty level of the examination to the presenter.

At step 310, the system may communicate the final examination document to the presenter. This step may be initiated by the provider, e.g., as in step 208 of method 200. Communication of the final examination document may include presenting an alert to the selected provider presenter to indicate that an exam is available for viewing. In some examples, the exam may be displayed automatically. In some examples, display of the exam may be initiated by presenter action, such as selecting an icon on the presenter's interface. As described above, the exam may include the list of selected exam components, possibly sorted into a logical order, as well as links to descriptive and/or instructional information (e.g., descriptions and/or demonstration videos) for each component.

At any suitable point in the process, the system may connect provider and presenter so as to allow modifications to or further communication of or about the exam document, based on real-time assessment and feedback. Communication may be via video, messaging, or another method, or by resending the exam document with modifications. In the latter case, the system may alert the presenter that modifications to the exam document are available.

One or more steps of method 300 may be repeated. For example, the provider may update the examination, in which case, the updated examination may be communicated to the presenter again via step 310.

D. Illustrative Method from the Presenter's Point of View

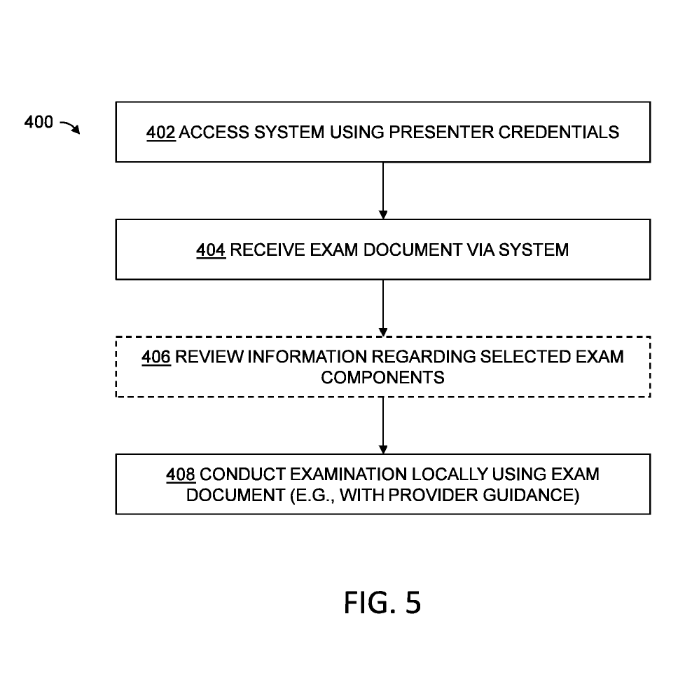

This section describes steps of an illustrative method 400 for utilizing system 10 and database 100 as a presenter 20; see FIG. 5. Aspects of system 10 and database system 100 may be utilized in the method steps described below. Where appropriate, reference may be made to previously described components and systems that may be used in carrying out each step, as well as other methods described herein. These references are for illustration, and are not intended to limit the possible ways of carrying out any particular step of the method.

FIG. 5 is a flowchart illustrating steps performed in an illustrative method, and may not recite the complete process or all steps of the method. FIG. 5 depicts multiple steps of a method, generally indicated at 400, which may be performed in conjunction with system 10, database system 100, and/or methods 200, and 300, according to aspects of the present disclosure. Although various steps of method 400 are described below and depicted in FIG. 5, the steps need not necessarily all be performed, and in some cases may be performed simultaneously or in a different order than the order shown.

Prior to performing method 400, the presenter may receive written, in-person, and/or online training on how to perform examinations and how to use system 10, and/or may observe examinations performed by another presenter using system 10. System 10 may be used for some or all of this training.

At step 402, the presenter may access the remote examination system, using presenter credentials (also referred to as logging in or signing in). These credentials are unique to each presenter/user, and provide limited access to the system. For example, presenters may not have permission to generate examinations, but may have permission to view an examination document generated by a provider, patient information, and/or information about exam components and/or the like. Step 402 may include sending a request for assistance, either generally or to a specific provider. The request for assistance may include information regarding the patient's condition (e.g., complaints, symptoms).

At step 404, the presenter may receive a prepared examination document via the system. For example, an icon may appear on the presenter's screen or GUI, indicating that an exam is available. Selecting this icon may cause the exam to be displayed to the presenter. In some examples, the prepared examination document may be prepared before the presenter signs in, and/or may be sent to the presenter before the presenter signs in (e.g., by sending the exam to the presenter's inbox or the equivalent). In some examples, the exam may not be sent until a connection is established between the provider and the presenter (i.e., with both parties on the system at the same time).

At step 406, the presenter may optionally review information regarding one or more of the exam components. For example, a component may be unfamiliar to the presenter, or may be infrequently performed, such that the presenter needs refresher training. Accordingly, the presenter may view the additional descriptive information and/or a demonstration video for the component(s) in question. Instructions and instructional videos may be stored for this purpose by system 10, and retrieved on demand.

At step 408, the presenter conducts the examination of the patient, using the examination document prepared by the provider, either independently or under the remote guidance of the provider (or a combination thereof). For example, when performed independently, the presenter may perform an examination step or component, report the results to the provider, and receive feedback. When performed under real-time guidance, the examination may be modified or halted by the provider, such as by providing an updated exam document in real time (e.g., based on patient assessment), or by stopping the process when a diagnosis has been sufficiently determined. One or more components of the examination may be performed before or after the supervised portion of the exam, and may be flagged accordingly by the provider. For example, the provider may designate selected exam components (e.g., blood pressure measurement) as “pre-exam” activities.

### E. Illustrative Data Processing System

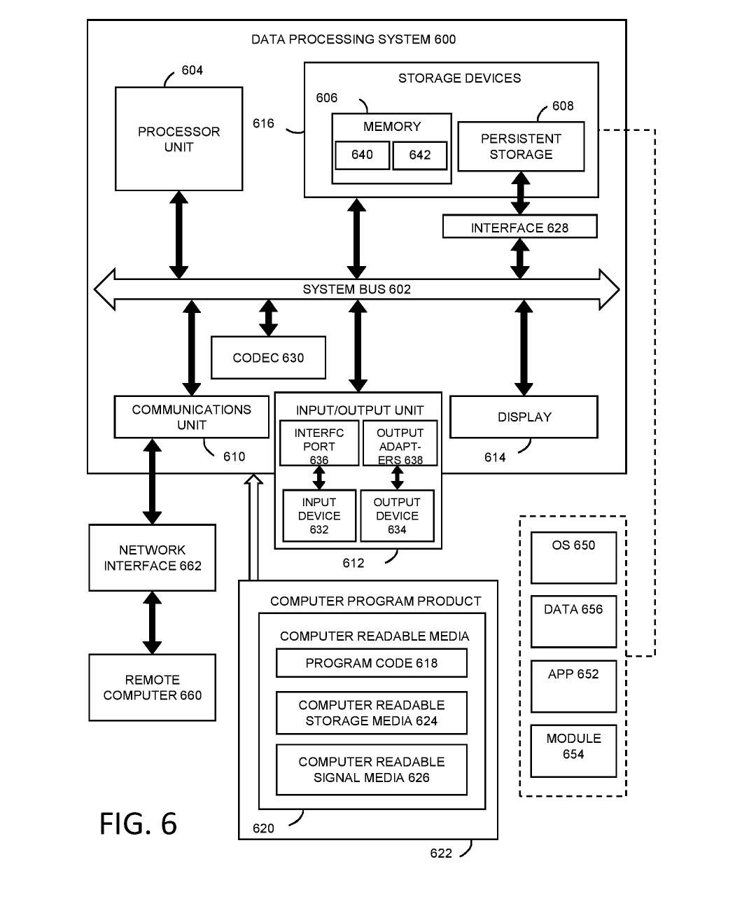

As shown in FIG. 6, this example describes a data processing system 600 (also referred to as a computer, computing system, and/or computer system) in accordance with aspects of the present disclosure. In this example, data processing system 600 is an illustrative data processing system suitable for implementing aspects of remote examination systems according to the present teachings. More specifically, in some examples, devices that are embodiments of data processing systems (e.g., smartphones, tablets, personal computers) may be utilized to access and interact with the system (e.g., system 10), may be utilized to execute the system (e.g., as servers on the network), and/or may be utilized to administer the system.

In this illustrative example, data processing system 600 includes a system bus 602 (also referred to as communications framework). System bus 602 may provide communications between a processor unit 604 (also referred to as a processor or processors), a memory 606, a persistent storage 608, a communications unit 610, an input/output (I/O) unit 612, a codec 630, and/or a display 614. Memory 606, persistent storage 608, communications unit 610, input/output (I/O) unit 612, display 614, and codec 630 are examples of resources that may be accessible by processor unit 604 via system bus 602.

Processor unit 604 serves to run instructions that may be loaded into memory 606. Processor unit 604 may comprise a number of processors, a multi-processor core, and/or a particular type of processor or processors (e.g., a central processing unit (CPU), graphics processing unit (GPU), etc.), depending on the particular implementation. Further, processor unit 604 may be implemented using a number of heterogeneous processor systems in which a main processor is present with secondary processors on a single chip. As another illustrative example, processor unit 604 may be a symmetric multi-processor system containing multiple processors of the same type.

Memory 606 and persistent storage 608 are examples of storage devices 616. A storage device may include any suitable hardware capable of storing information (e.g., digital information), such as data, program code in functional form, and/or other suitable information, either on a temporary basis or a permanent basis.

Storage devices 616 also may be referred to as computer-readable storage devices or computer-readable media. Memory 606 may include a volatile storage memory 640 and a non-volatile memory 642. In some examples, a basic input/output system (BIOS), containing the basic routines to transfer information between elements within the data processing system 600, such as during start-up, may be stored in non-volatile memory 642. Persistent storage 608 may take various forms, depending on the particular implementation.

Persistent storage 608 may contain one or more components or devices. For example, persistent storage 608 may include one or more devices such as a magnetic disk drive (also referred to as a hard disk drive or HDD), solid state disk (SSD), floppy disk drive, tape drive, Jaz drive, Zip drive, LS-60 drive, flash memory card, memory stick, and/or the like, or any combination of these. One or more of these devices may be removable and/or portable, e.g., a removable hard drive. Persistent storage 608 may include one or more storage media separately or in combination with other storage media, including an optical disk drive such as a compact disk ROM device (CD-ROM), CD recordable drive (CD-R Drive), CD rewritable drive (CD-RW Drive), and/or a digital versatile disk ROM drive (DVD-ROM). To facilitate connection of the persistent storage devices 608 to system bus 602, a removable or non-removable interface is typically used, such as interface 628.

Input/output (I/O) unit 612 allows for input and output of data with other devices that may be connected to data processing system 600 (i.e., input devices and output devices). For example, input device 632 may include one or more pointing and/or information-input devices such as a keyboard, a mouse, a trackball, stylus, touch pad or touch screen, microphone, joystick, game pad, satellite dish, scanner, TV tuner card, digital camera, digital video camera, web camera, and/or the like. These and other input devices may connect to processor unit 604 through system bus 602 via interface port(s) 636. Interface port(s) 636 may include, for example, a serial port, a parallel port, a game port, and/or a universal serial bus (USB).

Output devices 634 may use some of the same types of ports, and in some cases the same actual ports, as input device(s) 632. For example, a USB port may be used to provide input to data processing system 600 and to output information from data processing system 600 to an output device 634. Output adapter 638 is provided to illustrate that there are some output devices 634 (e.g., monitors, speakers, and printers, among others) which require special adapters. Output adapters 638 may include, e.g. video and sounds cards that provide a means of connection between the output device 634 and system bus 602. Other devices and/or systems of devices may provide both input and output capabilities, such as remote computer(s) 660. Display 614 may include any suitable human-machine interface or other mechanism configured to display information to a user, e.g., a CRT, LED, or LCD monitor or screen, etc.

Communications unit 610 refers to any suitable hardware and/or software employed to provide for communications with other data processing systems or devices. While communication unit 610 is shown inside data processing system 600, it may in some examples be at least partially external to data processing system 600. Communications unit 610 may include internal and external technologies, e.g., modems (including regular telephone grade modems, cable modems, and DSL modems), ISDN adapters, and/or wired and wireless Ethernet cards, hubs, routers, etc. Data processing system 600 may operate in a networked environment, using logical connections to one or more remote computers 660. A remote computer(s) 660 may include a personal computer (PC), a server, a router, a network PC, a workstation, a microprocessor-based appliance, a peer device, a smart phone, a tablet, another network note, and/or the like. Remote computer(s) 660 typically include many of the elements described relative to data processing system 600. Remote computer(s) 660 may be logically connected to data processing system 600 through a network interface 662 which is connected to data processing system 600 via communications unit 610. Network interface 662 encompasses wired and/or wireless communication networks, such as local-area networks (LAN), wide-area networks (WAN), and cellular networks. LAN technologies may include Fiber Distributed Data Interface (FDDI), Copper Distributed Data Interface (CDDI), Ethernet, Token Ring, and/or the like. WAN technologies include point-to-point links, circuit switching networks (e.g., Integrated Services Digital networks (ISDN) and variations thereon), packet switching networks, and Digital Subscriber Lines (DSL).

Codec 630 may include an encoder, a decoder, or both, comprising hardware, software, or a combination of hardware and software. Codec 630 may include any suitable device and/or software configured to encode, compress, and/or encrypt a data stream or signal for transmission and storage, and to decode the data stream or signal by decoding, decompressing, and/or decrypting the data stream or signal (e.g., for playback or editing of a video). Although codec 630 is depicted as a separate component, codec 630 may be contained or implemented in memory, e.g., non-volatile memory 642.

Non-volatile memory 642 may include read only memory (ROM), programmable ROM (PROM), electrically programmable ROM (EPROM), electrically erasable programmable ROM (EEPROM), flash memory, and/or the like, or any combination of these. Volatile memory 640 may include random access memory (RAM), which may act as external cache memory. RAM may comprise static RAM (SRAM), dynamic RAM (DRAM), synchronous DRAM (SDRAM), double data rate SDRAM (DDR SDRAM), enhanced SDRAM (ESDRAM), and/or the like, or any combination of these.

Instructions for the operating system, applications, and/or programs may be located in storage devices 616, which are in communication with processor unit 604 through system bus 602. In these illustrative examples, the instructions are in a functional form in persistent storage 608. These instructions may be loaded into memory 606 for execution by processor unit 604. Processes of one or more embodiments of the present disclosure may be performed by processor unit 604 using computer-implemented instructions, which may be located in a memory, such as memory 606.

These instructions are referred to as program instructions, program code, computer usable program code, or computer-readable program code executed by a processor in processor unit 604. The program code in the different embodiments may be embodied on different physical or computer-readable storage media, such as memory 606 or persistent storage 608. Program code 618 may be located in a functional form on computer-readable media 620 that is selectively removable and may be loaded onto or transferred to data processing system 600 for execution by processor unit 604. Program code 618 and computer-readable media 620 form computer program product 622 in these examples. In one example, computer-readable media 620 may comprise computer-readable storage media 624 or computer-readable signal media 626.

Computer-readable storage media 624 may include, for example, an optical or magnetic disk that is inserted or placed into a drive or other device that is part of persistent storage 608 for transfer onto a storage device, such as a hard drive, that is part of persistent storage 608. Computer-readable storage media 624 also may take the form of a persistent storage, such as a hard drive, a thumb drive, or a flash memory, that is connected to data processing system 600. In some instances, computer-readable storage media 624 may not be removable from data processing system 600.

In these examples, computer-readable storage media 624 is a physical or tangible storage device used to store program code 618 rather than a medium that propagates or transmits program code 618. Computer-readable storage media 624 is also referred to as a computer-readable tangible storage device or a computer-readable physical storage device. In other words, computer-readable storage media 624 is media that can be touched by a person.

Alternatively, program code 618 may be transferred to data processing system 600, e.g., remotely over a network, using computer-readable signal media 626. Computer-readable signal media 626 may be, for example, a propagated data signal containing program code 618. For example, computer-readable signal media 626 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 618 may be downloaded over a network to persistent storage 608 from another device or data processing system through computer-readable signal media 626 for use within data processing system 600. For instance, program code stored in a computer-readable storage medium in a server data processing system may be downloaded over a network from the server to data processing system 600. The computer providing program code 618 may be a server computer, a client computer, or some other device capable of storing and transmitting program code 618.

In some examples, program code 18 may comprise be an operating system (OS) 650. Operating system 650, which may be stored on persistent storage 608, controls and allocates resources of data processing system 600. One or more applications 652 take advantage of the operating system's management of resources via program modules 654, and program data 656 stored on storage devices 616. OS 650 may include any suitable software system configured to manage and expose hardware resources of computer 600 for sharing and use by applications 652. In some examples, OS 650 provides application programming interfaces (APIs) that facilitate connection of different type of hardware and/or provide applications 652 access to hardware and OS services. In some examples, certain applications 652 may provide further services for use by other applications 652, e.g., as is the case with so-called “middleware.” Aspects of present disclosure may be implemented with respect to various operating systems or combinations of operating systems.

The different components illustrated for data processing system 600 are not meant to provide architectural limitations to the manner in which different embodiments may be implemented. One or more embodiments of the present disclosure may be implemented in a data processing system that includes fewer components or includes components in addition to and/or in place of those illustrated for computer 600. Other components shown in FIG. 6 can be varied from the examples depicted. Different embodiments may be implemented using any hardware device or system capable of running program code. As one example, data processing system 600 may include organic components integrated with inorganic components and/or may be comprised entirely of organic components (excluding a human being). For example, a storage device may be comprised of an organic semiconductor.

In some examples, processor unit 604 may take the form of a hardware unit having hardware circuits that are specifically manufactured or configured for a particular use, or to produce a particular outcome or progress. This type of hardware may perform operations without needing program code 618 to be loaded into a memory from a storage device to be configured to perform the operations. For example, processor unit 604 may be a circuit system, an application specific integrated circuit (ASIC), a programmable logic device, or some other suitable type of hardware configured (e.g., preconfigured or reconfigured) to perform a number of operations. With a programmable logic device, for example, the device is configured to perform the number of operations and may be reconfigured at a later time. Examples of programmable logic devices include, a programmable logic array, a field programmable logic array, a field programmable gate array (FPGA), and other suitable hardware devices. With this type of implementation, executable instructions (e.g., program code 618) may be implemented as hardware, e.g., by specifying an FPGA configuration using a hardware description language (HDL) and then using a resulting binary file to (re)configure the FPGA.

In another example, data processing system 800 may be implemented as an FPGA-based (or in some cases ASIC-based), dedicated-purpose set of state machines (e.g., Finite State Machines (FSM)), which may allow critical tasks to be isolated and run on custom hardware. Whereas a processor such as a CPU can be described as a shared-use, general purpose state machine that executes instructions provided to it, FPGA-based state machine(s) are constructed for a special purpose, and may execute hardware-coded logic without sharing resources. Such systems are often utilized for safety-related and mission-critical tasks.

In still another illustrative example, processor unit 604 may be implemented using a combination of processors found in computers and hardware units. Processor unit 604 may have a number of hardware units and a number of processors that are configured to run program code 618. With this depicted example, some of the processes may be implemented in the number of hardware units, while other processes may be implemented in the number of processors.

In another example, system bus 602 may comprise one or more buses, such as a system bus or an input/output bus. Of course, the bus system may be implemented using any suitable type of architecture that provides for a transfer of data between different components or devices attached to the bus system. System bus 602 may include several types of bus structure(s) including memory bus or memory controller, a peripheral bus or external bus, and/or a local bus using any variety of available bus architectures (e.g., Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI)).

Additionally, communications unit 610 may include a number of devices that transmit data, receive data, or both transmit and receive data. Communications unit 610 may be, for example, a modem or a network adapter, two network adapters, or some combination thereof. Further, a memory may be, for example, memory 606, or a cache, such as that found in an interface and memory controller hub that may be present in system bus 602.

The flowcharts and block diagrams described herein illustrate the architecture, functionality, and operation of possible implementations of systems, methods, and computer program products according to various illustrative embodiments. In this regard, each block in the flowcharts or block diagrams may represent a module, segment, or portion of code, which comprises one or more executable instructions for implementing the specified logical function or functions. It should also be noted that, in some alternative implementations, the functions noted in a block may occur out of the order noted in the drawings. For example, the functions of two blocks shown in succession may be executed substantially concurrently, or the functions of the blocks may sometimes be executed in the reverse order, depending upon the functionality involved.

### F. Illustrative Distributed Data Processing System

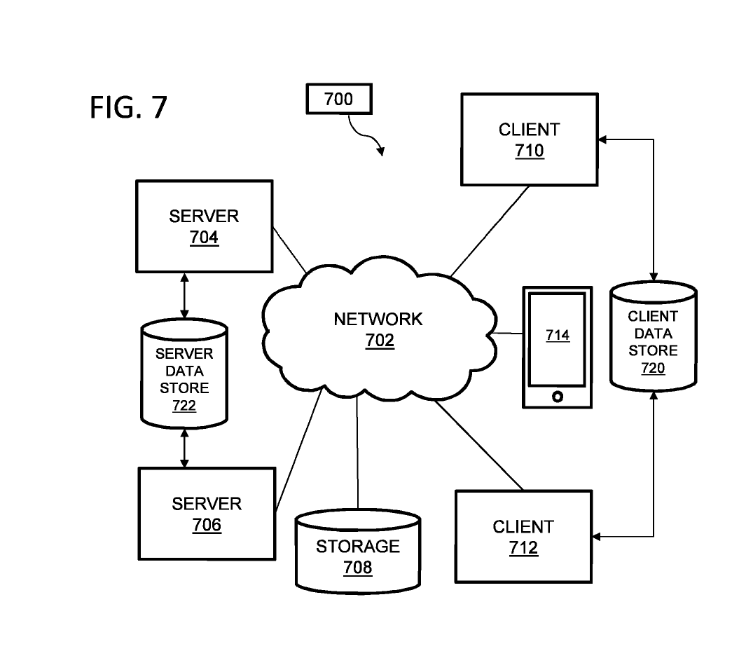

As shown in FIG. 7, this example describes a general network data processing system 700, interchangeably termed a computer network, a network system, a distributed data processing system, or a distributed network, in which may be included illustrative embodiments of remote examination systems, such as system 10. For example, system 10 may be implemented on a network, e.g., with a distributed architecture, such that data storage, program execution, site presentation, and/or communication may be performed over the network, and accessed by networked computers, such as provider and/or presenter's personal computers or portable devices.

It should be appreciated that FIG. 7 is provided as an illustration of one implementation and is not intended to imply any limitation with regard to environments in which different embodiments may be implemented. Many modifications to the depicted environment may be made.

Network system 700 is a network of devices (e.g., computers), each of which may be an example of data processing system 600, and other components. Network data processing system 700 may include network 702, which is a medium configured to provide communications links between various devices and computers connected within network data processing system 700. Network 702 may include connections such as wired or wireless communication links, fiber optic cables, and/or any other suitable medium for transmitting and/or communicating data between network devices, or any combination thereof.

In the depicted example, a first network device 704 and a second network device 706 connect to network 702, as do one or more computer-readable memories or storage devices 708. Network devices 704 and 706 are each examples of data processing system 600, described above. In the depicted example, devices 704 and 706 are shown as server computers, which are in communication with one or more server data store(s) 722 that may be employed to store information local to server computers 704 and 706, among others. However, network devices may include, without limitation, one or more personal computers, mobile computing devices such as personal digital assistants (PDAs), tablets, and smartphones, handheld gaming devices, wearable devices, tablet computers, routers, switches, voice gates, servers, electronic storage devices, imaging devices, media players, and/or other networked-enabled tools that may perform a mechanical or other function. These network devices may be interconnected through wired, wireless, optical, and other appropriate communication links.

In addition, client electronic devices 710 and 712 and/or a client smart device 714, may connect to network 702. Each of these devices is an example of data processing system 600, described above regarding FIG. 6. Client electronic devices 710, 712, and 714 may include, for example, one or more personal computers, network computers, and/or mobile computing devices such as personal digital assistants (PDAs), smart phones, handheld gaming devices, wearable devices, and/or tablet computers, and the like. In the depicted example, server 704 provides information, such as boot files, operating system images, and applications to one or more of client electronic devices 710, 712, and 714. Client electronic devices 710, 712, and 714 may be referred to as “clients” in the context of their relationship to a server such as server computer 704. Client devices may be in communication with one or more client data store(s) 720, which may be employed to store information local to the clients (e,g., cookie(s) and/or associated contextual information). Network data processing system 700 may include more or fewer servers and/or clients (or no servers or clients), as well as other devices not shown.

In some examples, first client electric device 710 may transfer an encoded file to server 704. Server 704 can store the file, decode the file, and/or transmit the file to second client electric device 712. In some examples, first client electric device 710 may transfer an uncompressed file to server 704 and server 704 may compress the file. In some examples, server 704 may encode text, audio, and/or video information, and transmit the information via network 702 to one or more clients.

Client smart device 714 may include any suitable portable electronic device capable of wireless communications and execution of software, such as a smartphone or a tablet. Generally speaking, the term “smartphone” may describe any suitable portable electronic device configured to perform functions of a computer, typically having a touchscreen interface, Internet access, and an operating system capable of running downloaded applications. In addition to making phone calls (e.g., over a cellular network), smartphones may be capable of sending and receiving emails, texts, and multimedia messages, accessing the Internet, and/or functioning as a web browser. Smart devices (e.g., smartphones) may also include features of other known electronic devices, such as a media player, personal digital assistant, digital camera, video camera, and/or global positioning system. Smart devices (e.g., smartphones) may be capable of connecting with other smart devices, computers, or electronic devices wirelessly, such as through near field communications (NFC), BLUETOOTH®, WiFi, or mobile broadband networks. Wireless connectively may be established among smart devices, smartphones, computers, and/or other devices to form a mobile network where information can be exchanged.

Data and program code located in system 700 may be stored in or on a computer-readable storage medium, such as network-connected storage device 708 and/or a persistent storage 608 of one of the network computers, as described above, and may be downloaded to a data processing system or other device for use. For example, program code may be stored on a computer-readable storage medium on server computer 704 and downloaded to client 710 over network 702, for use on client 710. In some examples, client data store 720 and server data store 722 reside on one or more storage devices 708 and/or 608.

Network data processing system 700 may be implemented as one or more of different types of networks. For example, system 700 may include an intranet, a local area network (LAN), a wide area network (WAN), or a personal area network (PAN). In some examples, network data processing system 700 includes the Internet, with network 702 representing a worldwide collection of networks and gateways that use the transmission control protocol/Internet protocol (TCP/IP) suite of protocols to communicate with one another. At the heart of the Internet is a backbone of high-speed data communication lines between major nodes or host computers. Thousands of commercial, governmental, educational and other computer systems may be utilized to route data and messages. In some examples, network 702 may be referred to as a “cloud.” In those examples, each server 704 may be referred to as a cloud computing node, and client electronic devices may be referred to as cloud consumers, or the like. FIG. 7 is intended as an example, and not as an architectural limitation for any illustrative embodiments.

### G. Additional Examples and Illustrative Combinations

This section describes additional aspects and features of remote examination systems and related methods, presented without limitation as a series of paragraphs, some or all of which may be alphanumerically designated for clarity and efficiency. Each of these paragraphs can be combined with one or more other paragraphs, and/or with disclosure from elsewhere in this application, in any suitable manner. Some of the paragraphs below expressly refer to and further limit other paragraphs, providing without limitation examples of some of the suitable combinations.

A0. A distributed data processing system for providing examination guidance remotely, the data processing system comprising:

a processor;

a memory in communication with the processor; and

a set of instructions stored in the memory and executable by the processor to:
query, in response to a search request from a first user at a first computer of the system, a data store containing a plurality of examination components, each examination component relating to at least one respective physiological system and to at least one respective diagnosis;
return a recommended subset of the examination components based on the search request;
generate, in response to selection of one or more of the examination components from the recommended subset by the first user, an examination procedure comprising the selected examination components; and
communicate the examination procedure to a second user at a second computer of the system.

A1. The system of A0, wherein the first computer has an established first communication link with a node of the system.

A2. The system of A1, wherein the second computer has an established second communication link with the node.

A3. The system of A1, wherein the node is a portion of a network-instantiated presentation layer.

A4. The system of A0, wherein generating the examination procedure comprises providing instructional information relating to each examination component.

A5. The system of A4, wherein the instructional information is stored in the system.

A6. The system of A0, wherein each of the examination components comprises information relating to a respective duration, and the examination procedure is configured to display a total expected duration based on the respective durations.

A7. The system of A0, wherein the first user is a provider and the second user is a presenter.

A8. The system of A0, the set of instructions being further executable to sort the examination components.

A9. The system of A8, wherein the examination components are sortable by physiological system.

A10. The system of A8, wherein the components are sortable according to a body map index.

A11. The system of A0, the set of instructions being further executable to:

automatically display pertinent summary information regarding the examination procedure.

A12. The system of A11, wherein the pertinent information comprises total duration.

A13. The system of A11, wherein the pertinent information comprises billing information.

A14. The system of A11, wherein the pertinent information comprises an overall difficulty of the examination.

B0. A method for remotely providing examination guidance, the method comprising:

establishing a first communication link between a first computer and a node in a computer network;

querying a database containing a plurality of examination components using the first computer, each of the examination components relating to at least one respective physiological system and to at least one respective medical diagnosis, by providing search criteria comprising a selected physiological system or a selected medical diagnosis or both;

narrowing a subset of the examination components returned from the database to generate an examination procedure comprising selected examination components from the subset;

establishing a second communication link between a second computer of the computer network and the node; and

communicating the examination procedure to the second computer via the node.

B1. The method of B0, wherein narrowing the subset of the examination components is performed, at least in part, automatically.

B2. The method of B1, wherein automatically narrowing the subset of the examination components includes comparing the subset to known patient information and excluding an examination component relating to the known patient information.

B3. The method of B0, further comprising:

automatically adding instructional information relating to performance of each of the examination components in the examination procedure.

B4. The method of B3, wherein the instructional information comprises video demonstration.

B5. The method of B3, wherein the instructional information comprises written instructions.

B6. The method of B3, wherein the instructional information comprises images.

B7. The method of B0, wherein the first computer corresponds to a provider and the second computer corresponds to a target presenter at a remote target facility.

C0. A method for remotely providing examination guidance, the method comprising:

establishing a first communication link between a first computer in a first facility and a network-instantiated presentation layer in a computer network;

querying a database containing a plurality of examination components using the first computer, each of the examination components relating to at least one respective physiological system and to at least one respective medical diagnosis, by providing search criteria comprising a selected physiological system or a selected medical diagnosis or both;

narrowing a subset of the examination components returned from the database to generate an examination procedure comprising selected examination components from the subset;

establishing a second communication link between a second computer of the computer network and the presentation layer, the second computer disposed in a second facility;

communicating the examination procedure to the second computer; and

providing a real-time communication channel between the first computer and the second computer via the presentation layer.

C1. The method of C0, further comprising:

guiding, from the first facility, a patient examination in the second facility, using the examination procedure and the real-time communication channel.

C2. The method of C0, wherein the communication channel comprises video chat.

C2a. The method of C0, wherein the communication channel comprises instant messaging.

C2b. The method of C0, wherein the communication channel comprises text messaging.

C3. The method of C0, wherein narrowing the subset of the examination components is performed, at least in part, automatically.

C4. The method of C3, wherein automatically narrowing the subset of the examination components includes integrating information relating to known best practices.

C5. The method of C3, wherein automatically narrowing the subset of the examination components includes comparing the subset to known patient information and excluding an examination component relating to the known patient information.

C6. The method of C0, further comprising:

automatically adding instructional information relating to performance of each of the examination components in the examination procedure.

C7. The method of C6, wherein the instructional information comprises video demonstration.

C8. The method of C0, wherein the first computer corresponds to a provider and the second computer corresponds to a target presenter at a remote target facility.

### Advantages, Features, Benefits

The different embodiments and examples of the remote examination system described herein provide several advantages over known solutions in the field of telehealth.

For example, illustrative embodiments and examples described herein allow clear and concise instructions to be passed from the provider to the presenter.

Additionally, and among other benefits, illustrative embodiments and examples described herein provide an updatable and repeatable data store of best practices and comprehensive listing of exam components, such that the provider need not rely on memory or habit for determining what examination steps should be performed.

Additionally, and among other benefits, illustrative embodiments and examples described herein allow selection and sorting of examination components for optimal efficiency and/or understanding.

Additionally, and among other benefits, illustrative embodiments and examples described herein provide built-in supplemental information regarding each exam component, such that the provider and/or presenter may refresh their understanding of the components in question, on demand.

Additionally, and among other benefits, illustrative embodiments and examples described herein provide a cloud-based solution, permitting providers and presenters to communicate examination procedures clearly using any suitable Internet-connected devices.

Additionally, and among other benefits, illustrative embodiments and examples described herein create a formalized yet straightforward method of communication between the provider and the presenter, ensuring an adequate level of care for the patient.

No known system or device can perform these functions, particularly in the field of remote examination of patients. Thus, the illustrative embodiments and examples described herein are particularly useful for providing health care solutions to rural and understaffed facilities, or facilities and locations that do not have ready access to on-site providers such as physicians and/or specialists. However, not all embodiments and examples described herein provide the same advantages or the same degree of advantage.

### Conclusion

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

What is claimed is:

1. A method for remotely providing examination guidance, the method comprising:
establishing a first communication link between a first computer and a node in a computer network;
querying a database containing a plurality of examination components using the first computer, each of the examination components relating to at least one respective physiological system and to at least one respective medical diagnosis, by providing search criteria comprising a selected physiological system or a selected medical diagnosis or both;
narrowing a subset of the examination components returned from the database to generate an examination procedure comprising selected examination components from the subset;
establishing a second communication link between a second computer of the computer network and the node; and
communicating the examination procedure to the second computer via the node.

2. The method of claim 1, wherein narrowing the subset of the examination components is performed, at least in part, automatically.

3. The method of claim 2, wherein automatically narrowing the subset of the examination components includes comparing the subset to known patient information and excluding an examination component relating to the known patient information.

4. The method of claim 1, further comprising:
automatically adding instructional information relating to performance of each of the examination components in the examination procedure.

5. The method of claim 4, wherein the instructional information comprises video demonstration.

6. The method of claim 1, wherein the first computer corresponds to a provider and the second computer corresponds to a target presenter at a remote target facility.

7. A method for remotely providing examination guidance, the method comprising:
establishing a first communication link between a first computer in a first facility and a network-instantiated presentation layer in a computer network;
querying a database containing a plurality of examination components using the first computer, each of the examination components relating to at least one respective physiological system and to at least one respective medical diagnosis, by providing search criteria comprising a selected physiological system or a selected medical diagnosis or both;
narrowing a subset of the examination components returned from the database to generate an examination procedure comprising selected examination components from the subset;
establishing a second communication link between a second computer of the computer network and the presentation layer, the second computer disposed in a second facility;
communicating the examination procedure to the second computer; and
providing a real-time communication channel between the first computer and the second computer via the presentation layer.

8. The method of claim 7, further comprising:
guiding, from the first facility, a patient examination in the second facility, using the examination procedure and the real-time communication channel.

9. The method of claim 7, wherein the communication channel comprises video chat.

10. The method of claim 7, wherein narrowing the subset of the examination components is performed, at least in part, automatically.

11. The method of claim 10, wherein automatically narrowing the subset of the examination components includes integrating information relating to known best practices.

12. The method of claim 10, wherein automatically narrowing the subset of the examination components includes comparing the subset to known patient information and excluding an examination component relating to the known patient information.

13. The method of claim 7, further comprising:
automatically adding instructional information relating to performance of each of the examination components in the examination procedure.

14. The method of claim 13, wherein the instructional information comprises video demonstration.

15. The method of claim 7, wherein the first computer corresponds to a provider and the second computer corresponds to a target presenter at a remote target facility.

16. A distributed data processing system for providing examination guidance remotely, the data processing system comprising:
a processor;
a memory in communication with the processor; and
a set of instructions stored in the memory and executable by the processor to:
query, in response to a search request from a first user at a first computer of the system, a data store containing a plurality of examination components, each examination component relating to at least one respective physiological system and to at least one respective diagnosis;
return a recommended subset of the examination components based on the search request;
generate, in response to selection of one or more of the examination components from the recommended subset by the first user, an examination procedure comprising the selected examination components; and
communicate the examination procedure to a second user at a second computer of the system.

17. The system of claim 16, wherein the first computer has an established first communication link with a node of the system, the second computer has an established second communication link with the node, and the node is a portion of a network-instantiated presentation layer.

18. The system of claim 16, wherein generating the examination procedure comprises providing instructional information relating to each examination component.

19. The system of claim 16, wherein each of the examination components comprises information relating to a respective duration, and the examination procedure is configured to display a total expected duration based on the respective durations.

20. The system of claim 16, the set of instructions being further executable to sort the examination components.
